(19) United States
(12) Patent Application Publication
(10) Pub. No.: US 2002/0168367 A1
(43) Pub. Date: Nov. 14, 2002

(54) Novel immunoadhesins for treating and preventing viral and bacterial diseases

(75) Inventors: James William Larrick, Woodside, CA (US); Keith Lynn Wycoff, Palo Alto, CA (US)

Correspondence Address: BROBECK, PHLEGER & HARRISON LLP, 12390 EL CAMINO REAL, SAN DIEGO, CA, 92130, US

(73) Assignee: PLANET BIOTECHNOLOGY INCORPORATED

(21) Appl. No.: 10/047,542

(22) Filed: Oct. 26, 2001

Related U.S. Application Data
(63) Continuation-in-part of application No. PCT/US01/13932, filed on Apr. 28, 2001.
(60) Provisional application No. 60/200,298, filed on Apr. 28, 2000.

(51) Int. Cl. A61K 39/40; A61K 39/07; A01H 1/00; C07K 16/46

(52) U.S. Cl. 424/164.1; 424/178.1; 800/288; 424/246.1; 530/388.4; 530/391.1

(57) ABSTRACT

Immunoadhesins active against infectious bacterial agents, e.g., the bacterium responsible for anthrax, are described and claimed. These chimeric molecules feature stabilizing immunoglobulin portions linked to bacterial proteins having affinity for host receptors. Methods of using these immunoadhesins are also desribed.

### RELATED APPLICATIONS

This application claims priority as a continuation-in-part application of International Application Ser. No. PCT/US01/13932, filed Apr. 28, 2001 in the name of Larrick and Wycoff, and entitled NOVEL IMMUNOADHESIN FOR THE PREVENTION OF RHINOVIRUS INFECTION, which in turn claims priority to U.S. Provisional Application Ser. No. 60/200,298, filed Apr. 28, 2000, and entitled the same. Each of these applications is herein incorporated by reference in its entirety, including all figures, drawings, and sequence listings.

### FIELD OF THE INVENTION

The present invention relates to immunoadhesins, fusions of the human anthrax toxin receptor protein and immunoglobulin, and the expression of immunoadhesins in plants. The therapeutic use of immunoadhesins for the treatment of human anthrax infection is also contemplated.

### BACKGROUND OF THE INVENTION

Applicants' previous application described the construction, purification, and use of chimeric immunoadhesin molecules, with examples and claims directed to treating or preventing viral infections and diseases. There is a need for similar agents for the treatment and prevention of bacterial infections and diseases, such as anthrax. The bioterrorism scare following Sep. 11, 2001 underscores this need.

The tripartite toxin secreted by Bacillus anthracis, the causative agent of anthrax, helps the bacterium evade the immune system and can kill the host during a systemic infection. Two components of the toxin enzymatically modify substrates within the cytosol of mammalian cells: oedema factor (OF) is an adenylate cyclase that impairs host defences through a variety of mechanisms including inhibiting phagocytosis; lethal factor (LF) is a zinc-dependent protease that cleaves mitogen-activated protein kinase kinase and causes lysis of macrophages. Protective antigen (PA), the third component, binds to a cellular receptor and mediates delivery of the enzymatic components to the cytosol. After binding to the cell-surface receptor, PA is cleaved into two fragments by a furin-like protease. The amino-terminal fragment, PA₂₀, dissociates into the medium, and this allows the carboxy-terminal fragment, PA63 to heptamerize and bind LF and OF. The resulting complexes of [PA₆₃]₇ with OF and/or LF are taken up into cells by receptor-mediated endocytosis and moved to a low-pH endosomal compartment. There, the acidic environment induces a conformational change in [PA₆₃]₇ that allows it to insert into the membrane and form a pore. This conversion promotes the translocation of bound OF and LF across the endosomal membrane to the cytosol.

The following documents may be useful in understanding the invention but are not admitted to be prior art to the invention:

Bäumlein H, Wobus U, Pustell J, Kafatos F C (1986) The legumin gene family: structure of a B type gene of Vicia faba and a possible legumin gene specific regulatory element. Nucl. Acids Res. 14: 2707-2713

Becker D, Kemper E, Schell J, Masterson R (1992) New plant binary vectors with selectable markers located proximal to the left T-DNA border. Plant Mol. Biol. 20: 1195-1197

Bradley K A, Mogridge J, Mourez M, Collier R J, Young J A T (2001) Identification of the cellular receptor for anthrax toxin. Nature 414: pre-publication

Chintalacharuvu K R, Raines M, Morrison S L (1994) Divergence of human alpha-chain constant region gene sequences. A novel recombinant alpha 2 gene. Journal of Immunology 152: 5299-5304

Depicker A, Stachel S, Dhaese P, Zambryski P, Goodman H M (1982) Nopaline synthase: transcript mapping and DNA sequence. J. Mol. Appl. Genet. 1: 561-573

Gielen J, De Beuckeleer M, Seurinck J, Deboeck F, De Greve H, Lemmers M, Van Montagu M, Schell J (1984) The complete nucleotide sequence of the TL-DNA of the Agrobacterium tumefaciens plasmid pTiAch5. Embo J 3: 835-46

Horsch R B, Fry J E, Hoffmann N L, Eichholtz D, Rogers S G, Fraley R T (1985) A simple and general method for transferring genes into plants. Science 227: 1229-1231

Ingelbrecht I, Breyne P, Vancompernolle K, Jacobs A, Van Montagu M, Depicker A (1991) Transcriptional interference in transgenic plants. Gene 109: 239-242

MacDonald M H, Mogen B D, Hunt AG (1991) Characterization of the polyadenylation signal from the T-DNA-encoded octopine synthase gene. Nucleic Acids Res 19: 5575-81

Mogen B D, MacDonald M H, Leggewie G, Hunt A G (1992) Several distinct types of sequence elements are required for efficient mRNA 3′ end formation in a pea rbcS gene. Mol Cell Biol 12: 5406-14

Ni M, Cui D, Einstein J, Narasimhulu S, Vergara C E, Gelvin S B (1995) Strength and tissue specificity of chimeric promoters derived from the octopine and mannopine synthase genes. Plant Journal 7: 661-676

Sawant S V, Singh P K, Gupta S K, Madnala R, Tuli R (1999) Conserved nucleotide sequences in highly expressed genes in plants. Journal of Genetics 78: 123-131

St Croix B, Rago C, Velculescu V, Traverso G, Romans K E, Montgomery E, Lal A, Riggins G J, Lengauer C, Vogelstein B, Kinzler K W (2000) Genes expressed in human tumor endothelium. Science 289: 1197-202.

Yamamoto Y Y, Tsuji H. Obokata J (1995) 5′-leader of a photosystem I gene in Nicotiana sylvestris, psaDb, contains a translational enhancer. J Biol Chem 270: 12466-70

### SUMMARY OF THE INVENTION

See claims and related applications incorporated by reference.

### BRIEF DESCRIPTION OF THE DRAWINGS

FIG. 1 shows the full nucleotide and amino acid sequence of the ATR-IgA2 fusion (an immunoadhesin).

FIG. 2 shows the sequence between the T-DNA borders of the plasmid pGPTV-kan-ocs-ATR-IgA2.

FIG. 3 shows the sequence between the T-DNA borders of the plasmid pGPTV-hpt-ocs-35SJ/SC.

### DETAILED DESCRIPTION

### EXAMPLES

1. Construction of Immunoadhesin Expression Cassettes

A cassette encoding a portion of the extracellular domains of human anthrax toxin receptor (ATR) is prepared by PCR cloning. Specifically, a fragment of 523 bp, encoding amino acids 44-216 (the so-called von Willebrand factor type A domain) is amplified from plasmid ATR (Bradley et al., 2001), or from plasmid TEM8 (St Croix et al., 2000) using the following oligonucleotide primers:

| | |
|---|---|
| 5′-GACCTGTACTTCATTTTGGACAAATCAGG-3′ | (SEQ ID |
| | NO: 1) |
| 5′-GAGCTCAAAATTGAGTGGATGATGCCTTGCAGAG-3′ | (SEQ ID |
| | NO: 2) |

The second primer (SEQ ID NO: 2) is designed to introduce a Sac I site at the 3′ end of the coding region of the ATR extracellular domain (solid underline). PCR is performed with Pfu polymerase (Stratagene) to reduce accumulation of errors. A second fragment of 124 bp, which includes a 5′ untranslated region and a plant signal peptide, is amplified from plasmid δATG-TOPO#4 (which is a PCR clone of a plant-optimized 5′ untranslated region and signal peptide in the Invitrogen cloning vector pCR4-TOPO), using the following oligonucleotide primers:

| | |
|---|---|
| 5′-GGTACCACTTCTCTCAATCCAACTTTC-3′ | (SEQ ID NO: 3) |
| 5′-GTCCAAAATGAAGTACAGGTCAGCCAAACTAGTAGAGGTGAACAAAAGC-3′ | (SEQ ID NO: 4) |

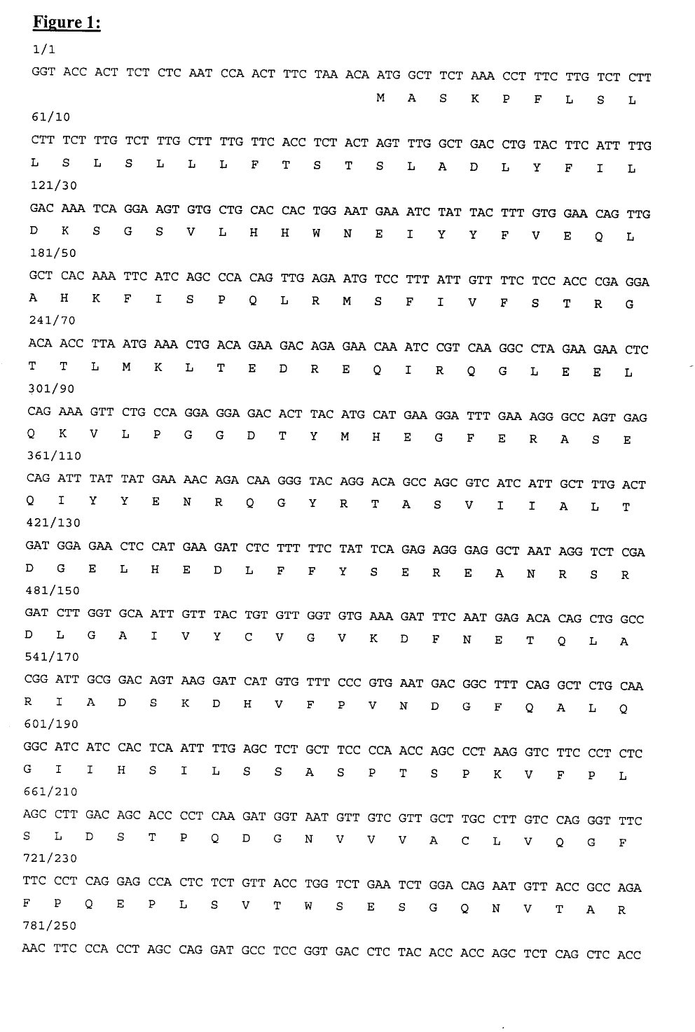

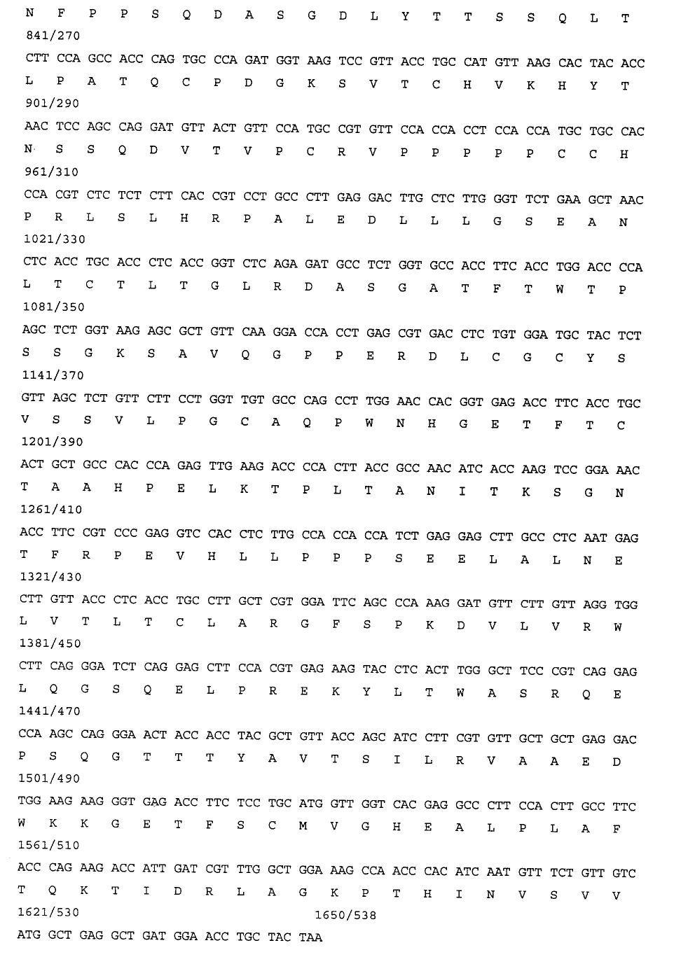

The first primer (SEQ ID NO: 3) is designed to introduce a Kpn I site at the 5′ end of the PCR fragment (solid underline). The two PCR fragments have 20 nt of complementary sequence (dotted underlines). The two PCR fragments are mixed together, and a fragment of 626 bp is amplified using SEQ ID NO: 3 and SEQ ID NO: 2. The resulting PCR fragment is cloned into the vector PCRScript (Stratagene), and sequenced before cloning between Kpn I and Sac I sites in the vector pMSP-coICAM, resulting in plasmid pMSP-ATR-IgA2. This results in a genetic fusion of the extracellular domain of ATR and the constant region of human IgA2. This human IgA2 constant region has been synthesized to use codons optimal for expression in tobacco cells. The full nucleotide and amino acid sequence of the ATR-IgA2 fusion (the immunoadhesin) is shown in FIG. 1. In the resulting construct, expression of the chimeric ATR-IgA2 molecule is under the control of the constitutive promoter “superMAS” (Ni et al., 1995) and the ags 3′ terminator region.

The entire expression cassette (promoter+ATR-IgA2+terminator) is removed from pMSP-ATR-IgA2 with the restriction enzyme Asc I, and cloned into the binary Agrobacterium Ti plasmid vector pGPTV-kan-ocs, resulting in plasmid pGPTV-kan-ocs-ATR-IgA2. The vector pGPTV-kan-ocs is derived from pGPTV-kan (Becker et al., 1992), which was modified in the following manner. The sequence between the Eco RI and Hind III sites of pGPTV-kan, including the entire uid A gene, was removed and replaced with the ocs 3′ terminator region (MacDonald et al., 1991) oriented toward the npt II gene, plus the restriction sites for Asc I and Sac I. The purpose of this terminator adjacent to the right border of the T-DNA is to eliminate transcriptional interference with the transgene due to transcription originating in the plant DNA outside of the right border (Ingelbrecht et al., 1991).

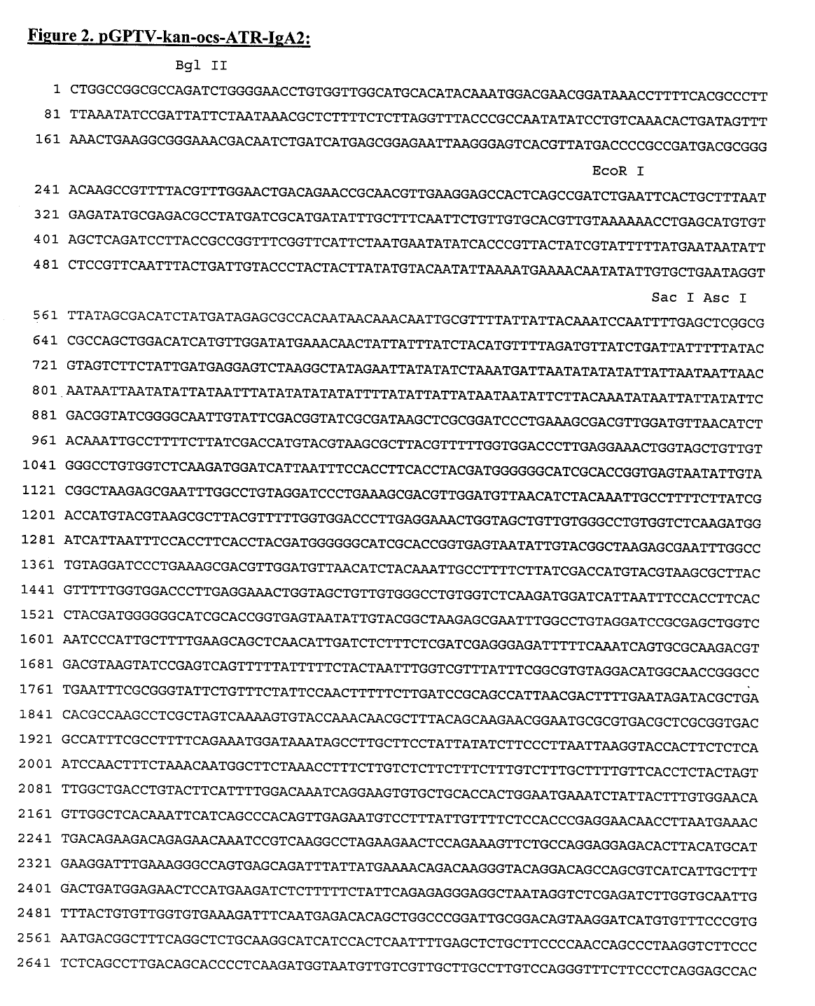

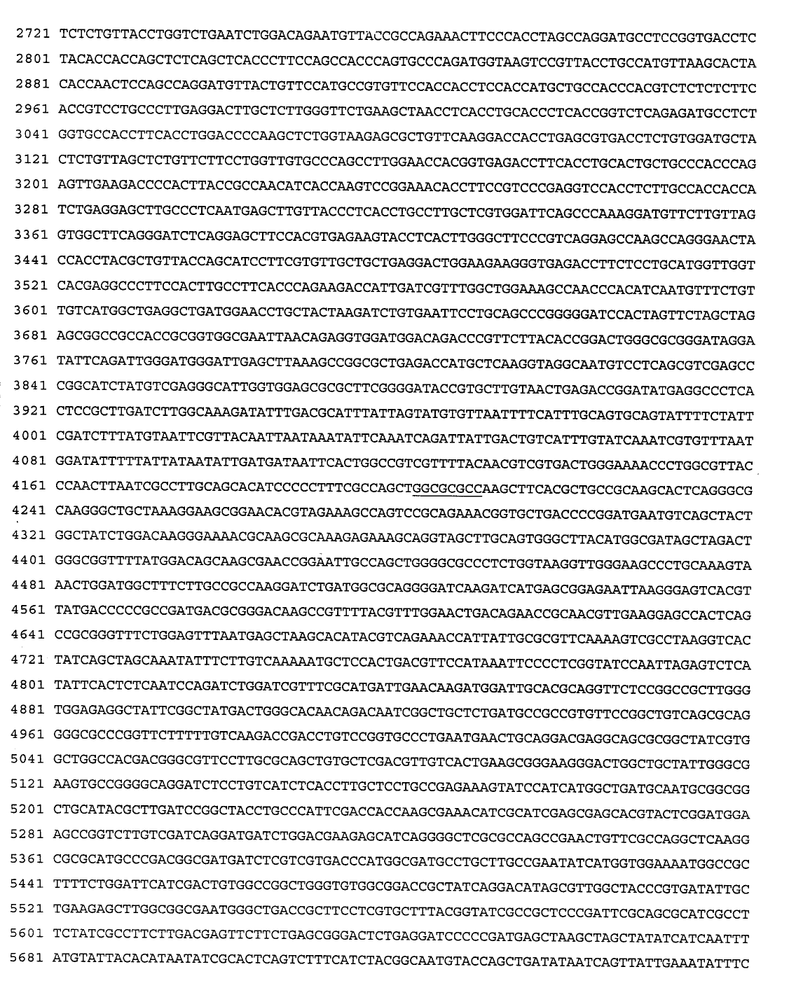

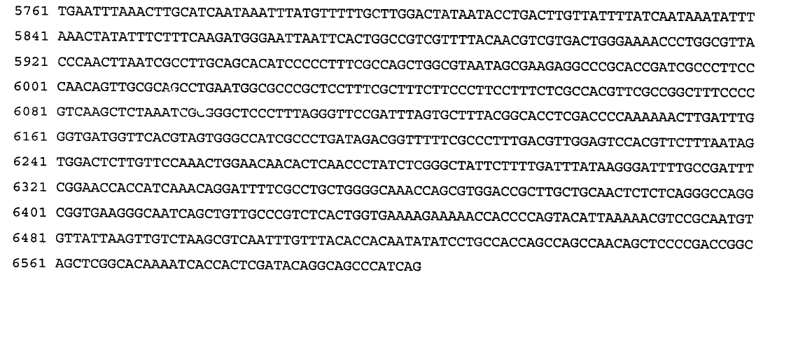

Sequence between the T-DNA borders of the plasmid pGPTV-kan-ocs-ATR-IgA2 is shown in FIG. 2. Sequence outside the left and right borders are as described (Becker et al., 1992). Nucleotides 18-187 represent the right T-DNA border. Nucleotides 311-630 represent the ocs 3′ terminator region. Nucleotides 927-1976 represent the superMAS promoter. Nucleotides 1990-2017 represent a 5′ untranslated region from the Nicotiana sylvestris psaDb gene (Yamamoto et al., 1995). The context around the initiation ATG (nucleotides 2012-2026) was designed to match that found in highly expressed plant genes (Sawant et al., 1999). Nucleotides 2018-2086 comprise a sequence encoding a modified version of the signal peptide of Vicia faba legumin (Bäumlein et al., 1986). Nucleotides 2087-2605 comprise a sequence encoding the von Willebrand factor type A domain of ATR (Bradley et al., 2001). Nucleotides 2606-3631 comprise a sequence encoding the human IgA2m(2) constant region (Chintalacharuvu et al., 1994). Nucleotides 3794-4108 derive from the agropine synthase (ags) terminator. Nucleotides 4530-4800 represent the NOS promoter (Depicker et al., 1982). Nucleotides 4835-5626 encode the npt II gene (conferring resistance to kanamycin). Nucleotides 5648-5870 are the polyadenylation signal from A. tumefactions gene 7 (Gielen et al., 1984). Nucleotides 6454-6602 represent the left T-DNA border.

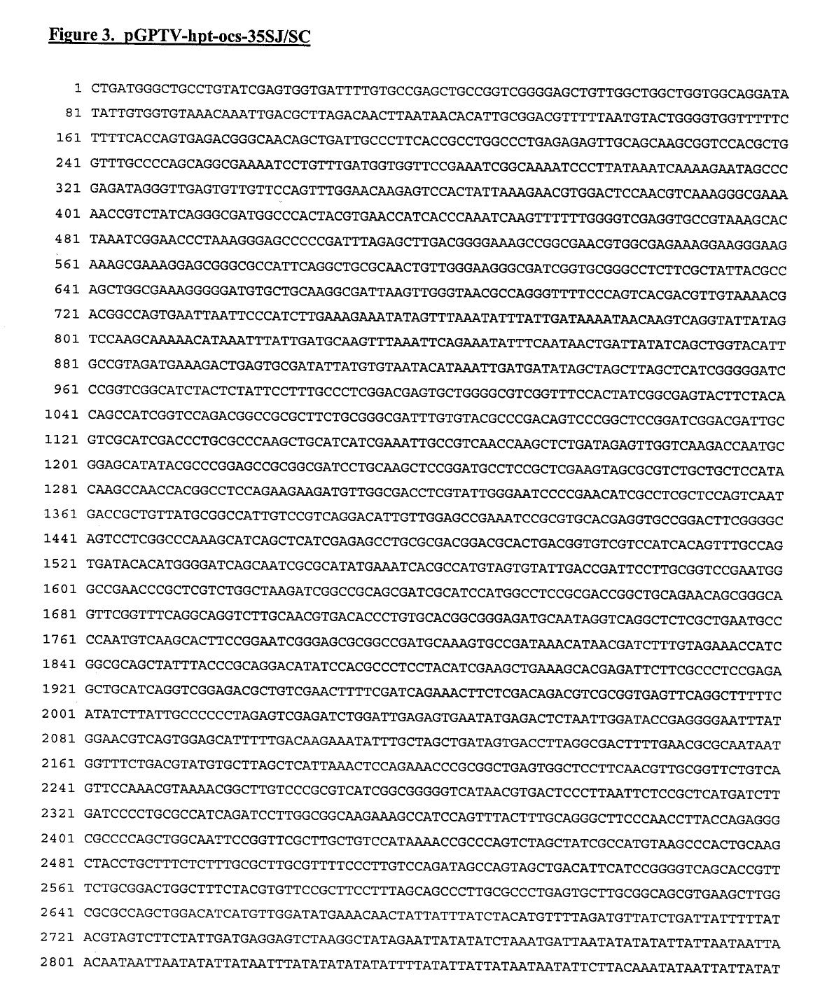

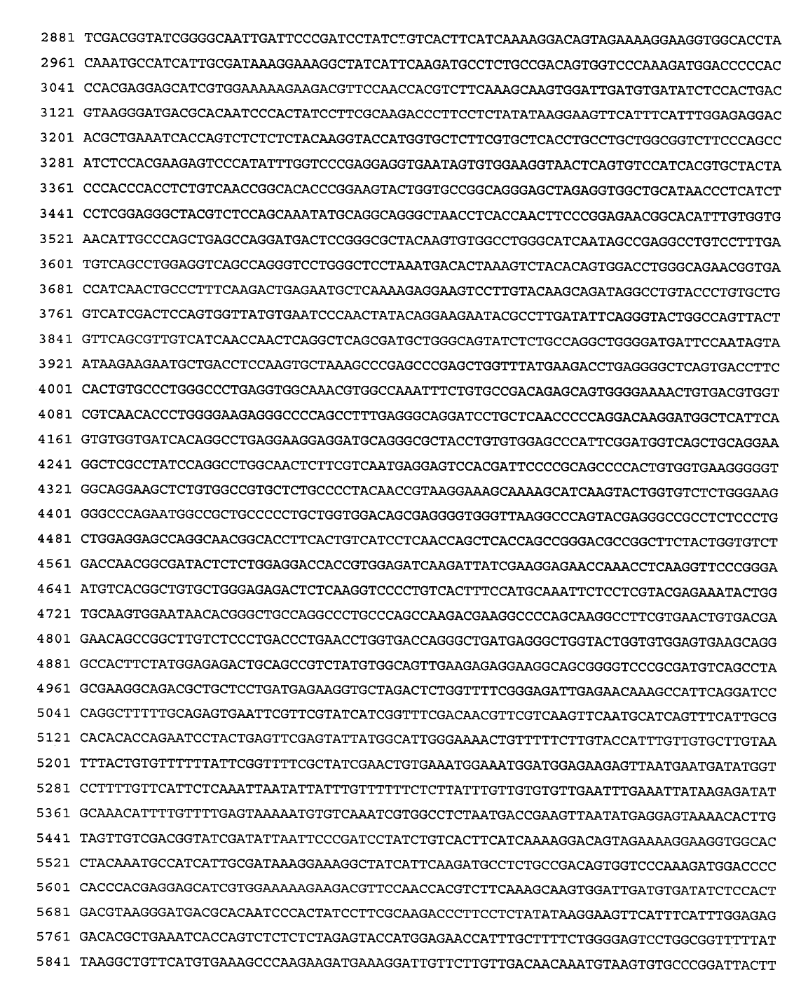

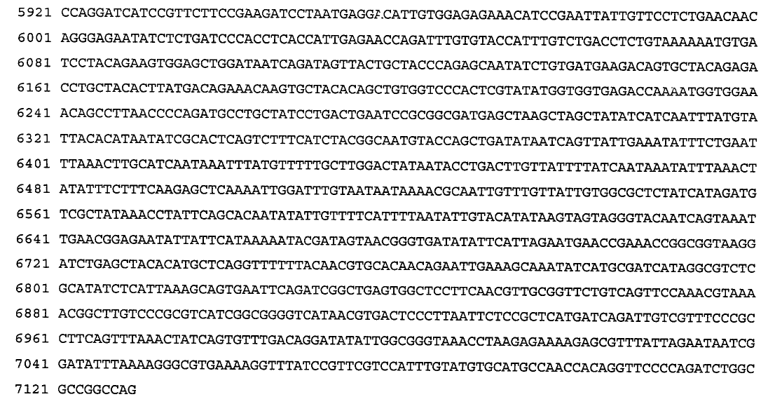

A construct for the expression in plants of human J chain and secretory component has also been developed. This construct, pGPTV-hpt-ocs-35SJ/SC, is based on the vector pGPTV-hpt-ocs, derived from pGPTV-hpt in the same manner as described for pGPTV-kan-ocs above. Sequence between the T-DNA borders of the plasmid pGPTV-hpt-ocs-35SJ/SC is shown in FIG. 3. Sequence outside the left and right borders are as described (Becker et al., 1992). Nucleotides 1-149 represent the left T-DNA border. Nucleotides 733-955 (complement) represent the polyadenylation signal from A. tumefactions gene 7 (Gielen et al., 1984). Nucleotides 980-2002 (complement) represent the hpt gene (conferring resistance to hygromycin). Nucleotides 2049-2318 (complement) represent the NOS promoter (Depicker et al., 1982). Nucleotides 2898-3230 represent the cauliflower mosaic virus (CaMV) 35S promoter driving expression of the human secretory component gene including it's native signal peptide (nucleotides 3236-5056), and nucleotides 5060-5445 represent the polyadenylation signal from the pea rbcS-E9 gene (Mogen et al., 1992). Nucleotides 5457-5788 represent a second copy of the CaMV 35S promoter driving expression of the human Joining (J) chain gene including it's native signal peptide (nucleotides 5797-6273), and nucleotides 6281-6494 represent the gene 7 terminator. Nucleotides 6501-6819 (complement) represent the ocs 3′ terminator region. Nucleotides 6944-7113 represent the right T-DNA border.

2. Plant Transformation and Immunoadhesin Expression in Plants

The expression cassettes described above are used to produce the assembled immunoadhesin in plants, via Agrobacterium-mediated transformation. Plasmids pGPTV-hpt-ocs-35SJ/SC and pGPTV-kan-ocs-ATR-IgA2 are introduced separately into A tumefaciens strain LBA4404. Overnight cultures of both strains are used for simultaneous “co-cultivation” with leaf pieces of tobacco, according to a standard protocol (Horsch et al., 1985). Transformed plant tissue is selected on regeneration medium containing both kanamycin (100 μg/mL) and hygromycin (25 μg/mL).

Plantlets that regenerate in the presence of antibiotic are screened for transgene expression. This is accomplished by preparing extracts of leaf tissue in phosphate buffered saline (PBS) and spotting clarified extracts on nitrocellulose paper. These “dot” blots are probed with alkaline-phosphatase-conjugated antisera specific for human IgA, J chain or secretory component. Plants that test positive on this first screen are subjected for further screens involving western blotting and PCR. The ATR-IgA2 immunoadhesin is expected to have a subunit MW of 59 kDa. Due to natural dimerization of the heavy chain constant region, dimers of ˜118 kDa are also expected to form. These dimers further dimerize within the plant cell in the presence of J chain, forming a molecule of ˜252 kDa. With the addition of secretory component, a molecular species of ˜320 kDa is observed.

The presence of a signal peptide in the chimeric heavy chain, J chain and secretory component constructs is important for assembly into a multimeric immunoadhesin. Upon translation of the mRNAs, signal peptide cleavage is predicted to deposit the each protein into the plant cell's endoplasmic reticulum (ER). Assembly into a multimeric immunoadhesin is expected to take place in the ER and golgi bodies, and the assembled molecule is then secreted from the cell.

3. Purification of Assembled Immunoadhesin

4. The Immunoadhesin Inhibits Toxin Action on Mammalian Cells

The expression cassettes described above are used to produce the assembled immunoadhesin, which is purified from plant extracts. The purified immunoadhesin is used to protect CHO-K1 cells from being killed in a simple bioassay. CHO-K1 cells have the receptor to which PA binds on their cell surfaces, but they are not sensitive to the toxin. They are killed when challenged with PA and LF_{N}-DTA, a fusion protein composed of the N-terminal 255 amino acids of LF linked to the catalytic A chain of diptheria toxin. This recombinant toxin exploits the same LF-PA-receptor interactions that are required for the binding and entry of the native LF and OF proteins. To test the protective effect of the immunoadhesin, CHO-K1 cells are mixed with an increasing amount of ATR-IgA2 in the presence of a constant (toxic) amount of PA and LF_{N}-DTA, and the subsequent effect on protein synthesis is measured. ATR-IgA2 is an effective inhibitor of toxin action, inhibiting toxin action at a lower molar concentration than soluble ATR.

5. The Immunoadhesin Inhibits Toxin Action in Human Subjects

The purified immunoadhesin is prepared in a pharmaceutically acceptable buffer and is administered to human subjects infected with Anthrax. The route of administration may be either as an inhaled aerosol or as an injection. Subjects in late stages of infection who would normally die are protected from toxin action by the immunoadhesin.

6. Construction of an Alternative Immunoadhesin Expression Cassette

A cassette encoding the entire extracellular portion of human ATR (amino acids 24-320) is prepared by PCR cloning. Specifically, a fragment of 878 bp is amplified from plasmid ATR (Bradley et al., 2001), or from plasmid TEM8 (St Croix et al., 2000) using the following oligonucleotide primers:

| | |
|---|---|
| 5′-GGGGGACGCAGGGAGGATGGGGGTCCAG-3′ | (SEQ ID NO: 5) |
| 5′-GAGCTCCCGTCAGAACAGTGTGTGGTGGTG-3′ | (SEQ ID NO: 6) |

The second primer (SEQ ID NO: 6) is designed to introduce a Sac I site at the 3′ end of the coding region of the ATR extracellular domain (solid underline). PCR is performed with Pfu polymerase (Stratagene) to reduce accumulation of errors. A second fragment of 121 bp, which includes a 5′ untranslated region and a plant signal peptide, is amplified from plasmid δATG-TOPO#4, using the following oligonucleotide primers:

| | |
|---|---|
| 5′-GGTACCACTTCTCTCAATCCAACTTTC-3′ | (SEQ ID NO: 3) |
| 5′-ATCCTCCCTGCGTCCCCCAGCCAAACTAGTAGAGGTGAACAAAAGC-3′ | (SEQ ID NO: 7) |

The first primer (SEQ ID NO: 3) is designed to introduce a Kpn I site at the 5′ end of the PCR fragment (solid underline). The two PCR fragments have 20 nt of complementary sequence (dotted underlines). The two PCR fragments are mixed together, and a fragment of 981 bp is amplified using SEQ ID NO: 3 and SEQ ID NO: 6. The resulting PCR fragment is cloned into a plant expression cassette to form a genetic fusion with human IgA2 in the same manner as the partial ATR extracellular domain (Example 1).

An alternate construction using this same method would amplify amino acids 41-227.

The foregoing examples are not limiting and merely representative of various aspects and embodiments of the present invention. All documents cited are indicative of the levels of skill in the art to which the invention pertains. The disclosure of each document is incorporated by reference herein to the same extent as if each had been incorporated by reference in its entirety individually, although none of the documents is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

Other embodiments are within the following claims.

We claim:

1. An immunoadhesin comprising a chimeric Anthrax Toxin Receptor protein, said Anthrax Toxin Receptor protein comprising:
an Anthrax Toxin Receptor protein linked to at least a portion of an immunoglobulin heavy chain; and
J chain and secretory component associated with said chimeric Anthrax Toxin Receptor protein.

2. The immunoadhesin of claim 1 wherein said Anthrax Toxin Receptor protein is comprised of the extracellular domain of Anthrax Toxin Receptor or any portion thereof.

3. The immunoadhesin of claim 1 wherein said immunoglobulin heavy chain is selected from the goup of
IgA, IgA1, IgA2, IgM, and chimeric immunoglobulin heavy chains.

4. The immunoadhesin of claim 1 comprising at least one additional chimeric Anthrax Toxin Receptor protein.

5. The immunoadhesin of claim 1 wherein said Anthrax Toxin Receptor protein is comprised of any portion of the extracellular domain of Anthrax Toxin Receptor protein; and said immunoglobulin heavy chain comprises at least a portion of an IgA2 heavy chain.

6. The immunoadhesin of claim 1 expressed in transgenic plants.

7. The immunoadhesin of claim 1 expressed in monocotyledonous plants.

8. The immunoadhesin of claim 1 expressed in dicotyledonous plants.

9. The immunoadhesin of claim 1 wherein all proteins are human.

10. The immunoadhesin of claim 1 expressed in heterologous cells derived from plants vertebrates, or invertebrates.

11. The immunoadhesin of claim 1 expressed in mammalian cells.

12. The immunoadhesin of claim 1 expressed in hairy root cultures

13. The immunoadhesin of claim 1 expressed in plant cells in tissue culture.

14. An immunoadhesin comprising a chimeric Anthrax Toxin Receptor protein, said Anthrax Toxin Receptor protein comprising: an Anthrax Toxin Receptor protein linked to at least a portion of an immunoglobulin heavy chain, wherein said immunoadhesin has plant-specific glycosylation.

15. The immunoadhesin of claim 14 wherein said immunoadhesin further comprises a J chain and secretory component associated with said chimeric Anthrax Toxin Receptor protein.

16. The immunoadhesin of claim 14 wherein said Anthrax Toxin Receptor protein is comprised of the extracellular domain of Anthrax Toxin Receptor or any portion thereof.

17. The immunoadhesin of cliam 14 wherein said immunoglobulin heavy chain is selected from the goup of IgA, IgA₁, IgA₂, IgG₁, IgG₂, IgG₃, IgG₄, IgD, IgE, IgM, and a chimeric immunoglobulin heavy chain.

18. The immunoadhesin of claim 14 comprising at least one additional chimeric Anthrax Toxin Receptor protein.

19. The immunoadhesin of claim 14 wherein said Anthrax Toxin Receptor protein is comprised of any portion of the extracellular domain of Anthrax Toxin Receptor protein;
and said immunoglobulin heavy chain comprises at least a portion of an IgA2 heavy chain.

20. The immunoadhesin of claim 14 wherein all proteins are human.

21. The immunoadhesin of claim 14 expressed in heterologous cells derived from plants vertebrates, or invertebrates.

22. The immunoadhesin of claim 14 expressed in hairy root cultures

23. The immunoadhesin of claim 14 expressed in plant cells in tissue culture.

24. The immunoadhesin of claim 14 expressed in transgenic plants.

25. The immunoadhesin of claim 14 expressed in monocotyledonous plants.

26. The immunoadhesin of claim 14 expressed in dicotyledonous plants.

27. A composition comprising an immunoadhesin and plant material, wherein said immunoadhesin comprises a chimeric Anthrax Toxin Receptor protein, said chimeric Anthrax Toxin Receptor protein linked to at least a portion of an immunoglobulin heavy chain.

28. The composition of claim 27 further comprising a J chain and secretory component with said chimeric Anthrax Toxin Receptor protein.

29. A composition of claim 27 wherein said chimeric Anthrax Toxin Receptor protein is comprised of any portion of the extracellular domain of Anthrax Toxin Receptor protein; and said immunoadhesin has plant-specific glycosylation.

30. A composition of claim 27 wherein said immunoglobulin heavy chain is selected from the goup of IgA, IgA₁, IgA₂, IgG₁, IgG₂, IgG₃, IgG₄, IgD, IgE, IgM, and a chimeric immunoglobulin heavy chain.

31. A composition of claim 27 comprising at least one additional chimeric Anthrax Toxin Receptor protein.

32. A composition of claim 27 wherein said Anthrax Toxin Receptor protein is comprised of any portion of the extracellular domain of Anthrax Toxin Receptor protein; and said immunoglobulin heavy chain comprises at least a portion of an IgA2 heavy chain.

33. A method for reducing the binding of protective antigen (PA) of Bacillus anthracis to host cells susceptible to damage by anthrax toxin, said method comprising: contacting PA with an immunoadhesin of claim 1, 14 or 27, and wherein said immunoadhesin binds to PA and reduces the toxic activity thereof.

34. A method for reducing mortality and morbidity due to anthrax toxin, said method comprising: contacting PA with an immunoadhesin of claim 1, 14 or 27, and wherein said immunoadhesin binds to PA and reduces the toxic activity thereof.

35. A method for reducing mortality and morbidity due to anthrax toxin in a human subject, said method comprising: administering to said subject an effective amount of an immunoadhesin of claim 1, 14 or 27, and wherein said immunoadhesin binds to PA and reduces the toxic activity thereof.

36. A pharmaecutical composition comprising an immunoadhesin of claim 1, 14 or 27 in a pharmaceutically acceptable buffer.

37. An expression vector comprising a gene encoding a chimeric anthrax toxin receptor protein operatively linked to a plant promoter, said chimeric anthrax toxin receptor protein linked to at least a portion of an immunoglobulin heavy chain.
